(19) United States
(12) Patent Application Publication
(10) Pub. No.: US 2025/0135084 A1
(43) Pub. Date: May 1, 2025

(54) ASPIRATION AND INFUSION CONNECTION INDICATOR LIGHT FOR VITREORETINAL AND CATARACT SYSTEM

(71) Applicant: Alcon Inc., Fribourg (CH)

(72) Inventors: Eric LEE, Irvine, CA (US); Marc NESTOR, Aliso Viejo, CA (US); Andrew ZOOLAKIS, Carlsbad, CA (US); William MONTELEONE, Aliso Viejo, CA (US); Jeffrey Jikang CHUN, Ladera Ranch, CA (US); David Mervyn ANDREWS, San Clemente, CA (US); Brandon L. SPOELSTRA, Trabuco Canyon, CA (US)

(21) Appl. No.: 18/889,066

(22) Filed: Sep. 18, 2024

Related U.S. Application Data
(60) Provisional application No. 63/594,744, filed on Oct. 31, 2023.

(51) Int. Cl. A61M 1/00; A61B 17/00; A61F 9/007

(52) U.S. Cl. CPC A61M 1/72; A61F 9/00736; A61M 1/74; A61M 1/77; A61B 2017/00199; A61B 2217/005; A61B 2217/007; A61M 2205/583; A61M 2205/6072

(57) ABSTRACT

Certain embodiments disclosed herein provide an ophthalmic surgical system. The system includes a fluidics module and a controller. The fluidic module includes a surgical cassette bay and one or more indicator lights. The one or more indicator lights correspond to one or more fluidic ports on the fluidics module. The controller includes a memory including executable instructions and a processor. The processor is in data communication with the memory. The processor is configured to execute the instructions to cause the system to: determine that a surgical cassette is coupled to the surgical cassette bay; determine a type of the surgical cassette; illuminate at least one of the one or more indicator lights based on the determined type of the surgical cassette; and extinguish the at least one of the one or more indicator lights upon receiving an input indicating that a fluidic tubing is fluidically coupled with the corresponding fluidic port.

### CROSS-REFERENCE TO RELATED APPLICATIONS

This application claim benefit of and priority to U.S. Provisional Patent Application No. 63/594,744, filed Oct. 31, 2023, which is hereby assigned to the assignee hereof and hereby expressly incorporated by reference in its entirety as if fully set forth below and for all applicable purposes.

### INTRODUCTION

In a wide variety of medical procedures, the severance and removal of portions of the eye is required. For example, in vitreoretinal surgery, such as vitrectomy, a microsurgical instrument is inserted into a patient's eye through one or more incisions made in the eye to cut and remove the vitreous material within. An aspiration tube, which may be integrated with the microsurgical instrument, is used to remove the vitreous material from the eye of the patient. Meanwhile, an infusion tube is used to provide replacement fluids, such as silicone oil or saline, to the eye of the patient in order to maintain intraocular pressure (IOP).

Typically, one or more of the microsurgical instrument, the aspiration tube, and the infusion tube are operably coupled to a surgical console during the procedure. Such devices and fluidic lines are usually connected to the surgical console prior to performance of the surgical procedure during one or more setup operations of the surgical console. Embodiments described herein are directed towards facilitating the setup of a surgical console for vitreoretinal surgeries, and/or other ophthalmic surgeries.

### SUMMARY

The present disclosure relates generally to ophthalmic surgical system, and more specifically, to methods and systems for preparing ophthalmic surgical systems for surgery.

Certain embodiments of the present disclosure provide an ophthalmic surgical system. The system includes a fluidics module and a controller. The fluidic module includes a surgical cassette bay and one or more indicator lights. The one or more indicator lights correspond to one or more fluidic ports on the surgical cassette. The controller includes a memory including executable instructions and a processor. The processor is in data communication with the memory. The processor is configured to execute the instructions to cause the system to: determine that a surgical cassette is coupled to the surgical cassette bay; determine a type of the surgical cassette; and illuminate at least one of the one or more indicator lights based on the determined type of the surgical cassette.

Certain embodiments of the present disclosure provide a method of configuring a surgical console for an ophthalmic surgical procedure. The method of configuring a surgical console for an ophthalmic surgical procedure, comprising: determining that a surgical cassette is coupled to a surgical cassette bay; determining a type of the surgical cassette; and illuminating at least one of one or more indicator lights based on the determined type of the surgical cassette.

The following description and the related drawings set forth in detail certain illustrative features of one or more embodiments, including those described above.

### BRIEF DESCRIPTION OF THE DRAWINGS

So that the manner in which the above-recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope and may admit to other equally effective embodiments.

FIG. 1 illustrates an example surgical console for performing ophthalmic surgical procedures, in accordance with certain embodiments of the present disclosure.

FIG. 2A illustrates a fluidic module of the surgical console without a surgical cassette, in accordance with certain embodiments of the present disclosure.

FIG. 2B illustrates the fluidic module with the surgical cassette received in a surgical cassette bay of the surgical console, in accordance with certain embodiments of the present disclosure.

FIG. 2C illustrates the fluidic module with fluidic tubing received in fluidic ports of the surgical console, in accordance with certain embodiments of the present disclosure.

FIG. 3A illustrates an exploded perspective view of the fluidic module panel, in accordance with certain embodiments of the present disclosure.

FIG. 3B illustrates an exploded perspective of a rear panel of the fluidics module, in accordance with certain embodiments of the present disclosure.

FIG. 3C illustrates the rear portion of the second component of the rear panel of the fluidics module, in accordance with certain embodiments of the present disclosure.

FIG. 3D illustrates a segment of the rear portion, in accordance with certain embodiments of the present disclosure.

FIG. 4 illustrates a schematic of the controller, in accordance with certain embodiments of the present disclosure.

FIG. 5 illustrates a flow diagram of a method of configuring a surgical console for an ophthalmic surgical procedure, in accordance with certain embodiments of the present disclosure.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

In the following description, details are set forth by way of example to facilitate an understanding of the disclosed subject matter. It should be apparent to a person of ordinary skill in the field, however, that the disclosed implementations are exemplary and not exhaustive of all possible implementations. Thus, it should be understood that reference to the described examples is not intended to limit the scope of the disclosure. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one implementation may be combined with the features, components, and/or steps described with respect to other implementations of the present disclosure.

Particular embodiments disclosed herein generally relate to ophthalmic surgical systems, and more specifically, to methods and systems for preparing the ophthalmic surgical systems for surgery.

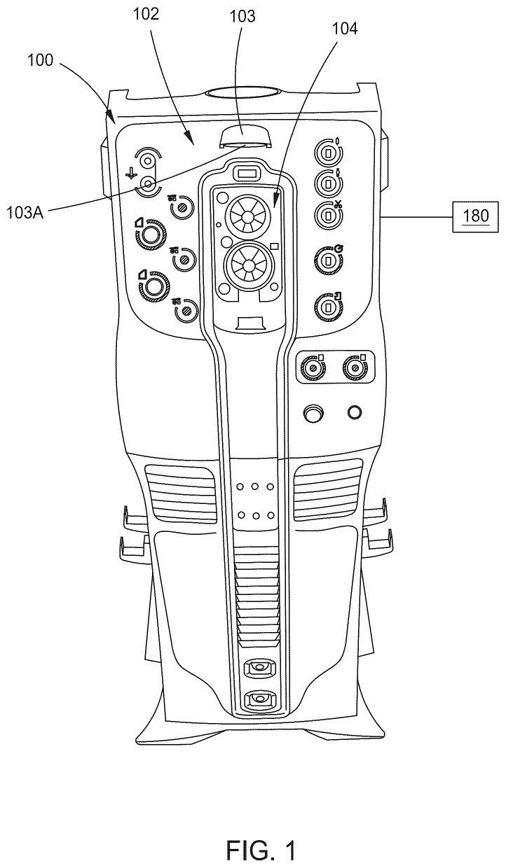

Aspects of the present disclosure provide for more efficient preparation of an ophthalmic surgical system. The ophthalmic surgical system includes one or more indicator lights that correspond to fluidic ports, such as fluidic ports on a surgical cassette. The one or more indicator lights indicate the fluidic ports that will receive a fluidic tubing, based on executable instructions stored in the memory of a controller of the ophthalmic surgical system. In certain embodiments, when the fluidic port has received the fluidic tubing, the indicator light may extinguish. The ophthalmic surgical system increases the efficiency and ease of set up of the ophthalmic surgical system, thereby improving overall procedural efficiency. In certain embodiments, the ophthalmic surgical system also facilitates improved visibility for a user in dimly lit environments, without introducing excess light to the surgical environment. Such embodiments are described below in further detail in conjunction with FIGS. 1, 2A-2C, 3A-3C, 4 and 5.

FIG. 1 illustrates an example surgical console (e.g., an ophthalmic surgical system 100) for performing ophthalmic surgical procedures. The ophthalmic surgical system 100 includes a fluidic module 102 and a controller 180. The fluidic module 102 includes a surgical cassette bay 104, a task light housing 103, and a fluidic module panel 114. The task light housing 103 is configured to include a task light 103A. The task light housing 103 may include a molded plastic with an aluminum-coated mirrored surface to direct the light from the task light 103A toward the surgical cassette bay 104. The task light 103A may include a diffusive window. The diffusive window may spread and soften the light, as well as keep dust and debris out of the ophthalmic surgical system 100.

The task light 103A may include one or more lights. The task light 103A is configured to illuminate the surgical cassette bay 104 and a portion of the fluidic module 102. In some embodiments, a first light of the one or more lights may illuminate an area directly downward from the task light housing 103, a second light of the one or more lights may illuminate an area downward and to the left of the task light housing 103, and a third light of the one or more lights may illuminate an area downward and to the right of the task light housing 103. The first light, second light, and third light may be surface mounted LEDs on a first printed circuit board assembly (PCBA). The first light, second light, and third light may be configured to operate independently of each other.

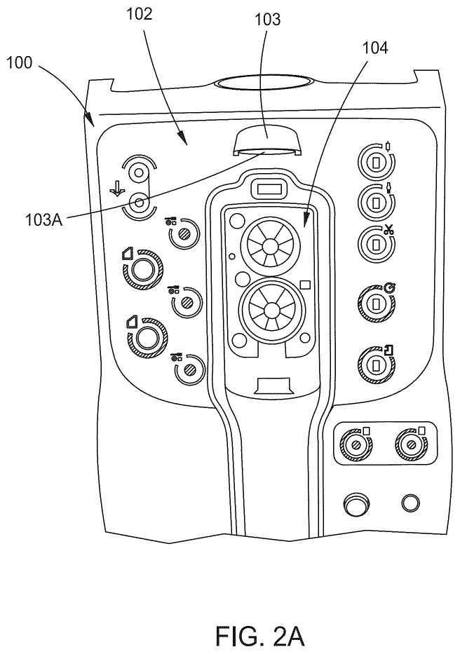

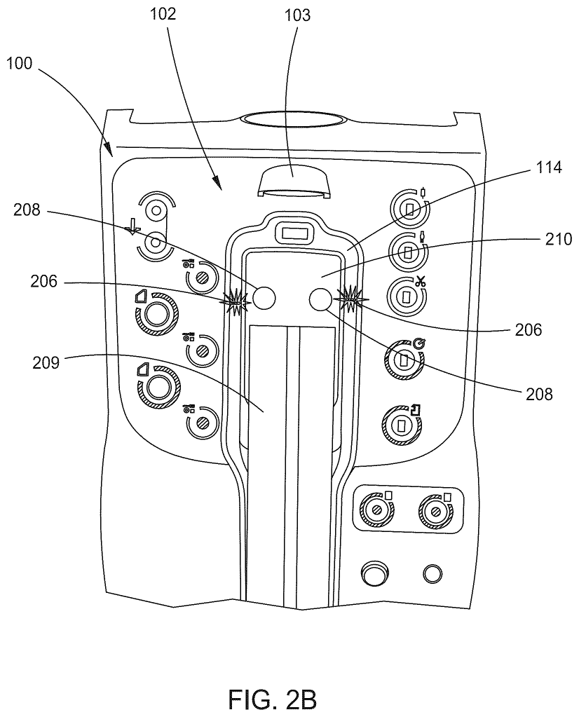

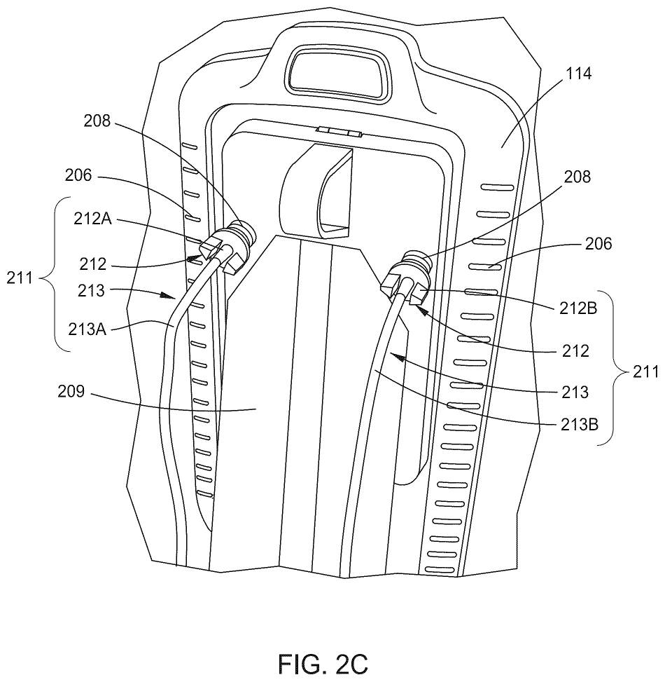

FIG. 2A illustrates the fluidic module 102 without a surgical cassette 210. FIG. 2B illustrates the fluidic module 102 with the surgical cassette 210 received in the surgical cassette bay 104. The surgical cassette bay 104 is configured to receive the surgical cassette 210. The surgical cassette 210 includes one or more fluidic ports 208, a fluid repository 209, and an identifier. The identifier may be a bar code, a QR (Quick Response) code, RFID (radio frequency identification) tag, or other identifying feature of the surgical cassette 210. The controller 180 of the ophthalmic surgical system 100 may be configured to scan the identifier to determine a type of the surgical cassette 210.

One or more indicator lights 206 correspond to the one or more fluidic ports 208. The fluidic module panel 114 is configured to house the one or more indicator lights 206. The fluidic module panel 114 surrounds the surgical cassette bay 104. The fluidic module panel 114 may include a second PCBA that includes the one or more indicator lights 206. The one or more indicator lights 206 may be light emitting diodes (LEDs).

The one or more indicator lights 206 may be configured to be “dead-fronted”, e.g., when the one or more indicator lights 206 are not illuminated, the area of the fluidic module 102 having the one or more indicator lights 206 are indistinguishable from the surrounding area of the fluidic module 102. The use of dead-fronted indicator lights 206 enables the ophthalmic surgical system 100 to be used for procedures in which the one or more indicator lights are not needed. Excess light in surgical environments may cause distractions. Therefore, the reduction of excess light may be beneficial to the use of the ophthalmic surgical system 100. Further, the dead-fronted indicator light 206 reduces the likelihood of confusion during set up by eliminating the illumination of indicator lights 206 corresponding to unnecessary fluidic ports 208.

FIG. 2C illustrates the fluidic module 102 with fluidic tubing 211 received in the fluidic ports 208. The one or more fluidic ports 208 are configured to receive the fluidic tubing 211. The fluidic tubing 211 may include a fluidic tube 213 and a fluidic tube connector 212. In some embodiments, the fluidic tubing 211 may include an aspiration tube connector 212A configured to connect an aspiration tube 213A to the fluidic port 208 and an infusion tube connector 212B configured to connect an infusion tube 213B to a fluidic port 208. The aspiration tube 211A is configured to remove vitreous material from the patient's eye to the fluid repository 209. The infusion tube 211B is configured to provide replacement fluid to the patient's eye from a replacement fluid source. The replacement fluid may be a silicone oil or a saline solution (e.g., balanced saline solution (BSS)). The replacement fluid replaces intraocular fluid that may be removed with the emulsified and ablated tissue in order to maintain the intraocular pressure of the patient's eye.

In some embodiments, components of the fluidic tubing 211, such as the fluidic tube 213 or corresponding fluidic tube connector 212, may be color coded based on the function of the tubing. For example, the aspiration tube connector 212A may be blue while the infusion tube connector 212B may be green. In addition, the one or more indicator lights 206 may each be color coded to correspond with a respective fluidic tube connector (e.g., aspiration tube connector 212A or infusion tube connector 212B) the indicator light 206 is configured to indicate or represent. In other words, each indicator lights 206 may have the same or similar color to a respective fluidic tube connector 212, e.g., the fluidic port 208 corresponding to the aspiration tube connector 212A may correspond with a blue indicator light, while the fluidic port 208 corresponding to the infusion tube connector 212B may correspond with a green indicator light.

In some embodiments, the fluidic ports 208 are configured to accept only the corresponding fluidic tube connector 212, e.g., the fluidic port 208 corresponding to the aspiration tube connector 212A is configured to accept the aspiration tube connector 212A and is configured to be incompatible with the infusion tube connector 212B. In another example, white LED indicator lights 206 may indicate the patent eye level (PEL) pressure setting. The one or more indicator lights 206 may have a shape, such as an arrow shape, in some embodiments in order to further assist in preparation of the ophthalmic surgical system 100 for surgery.

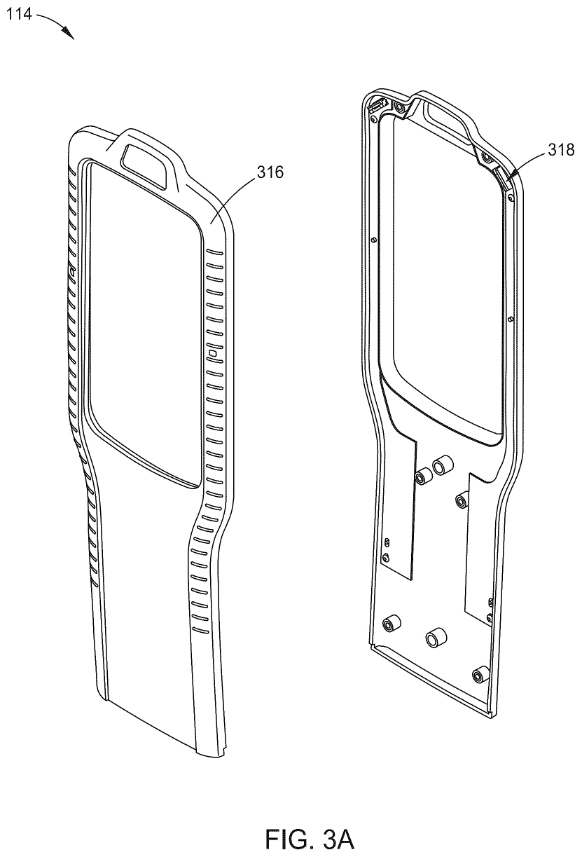

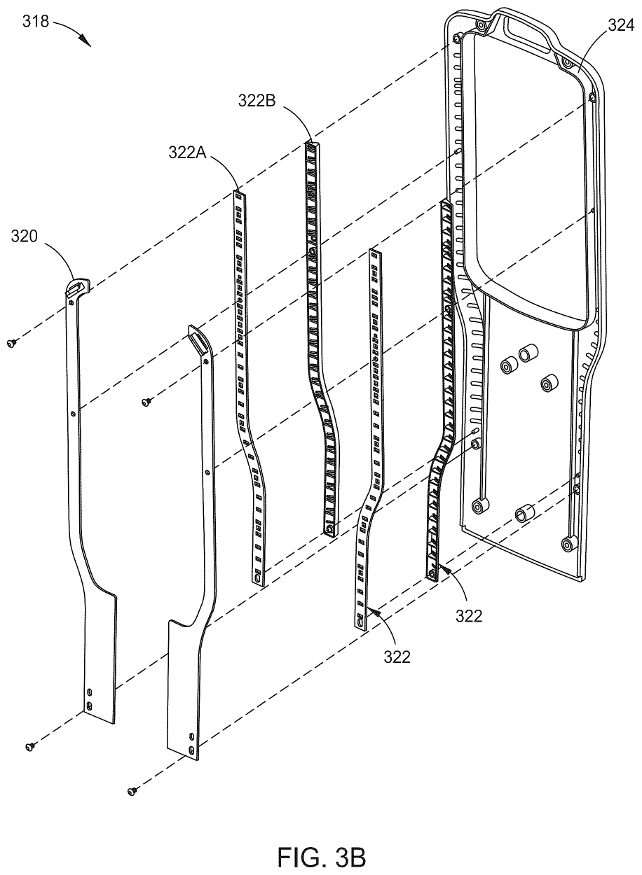

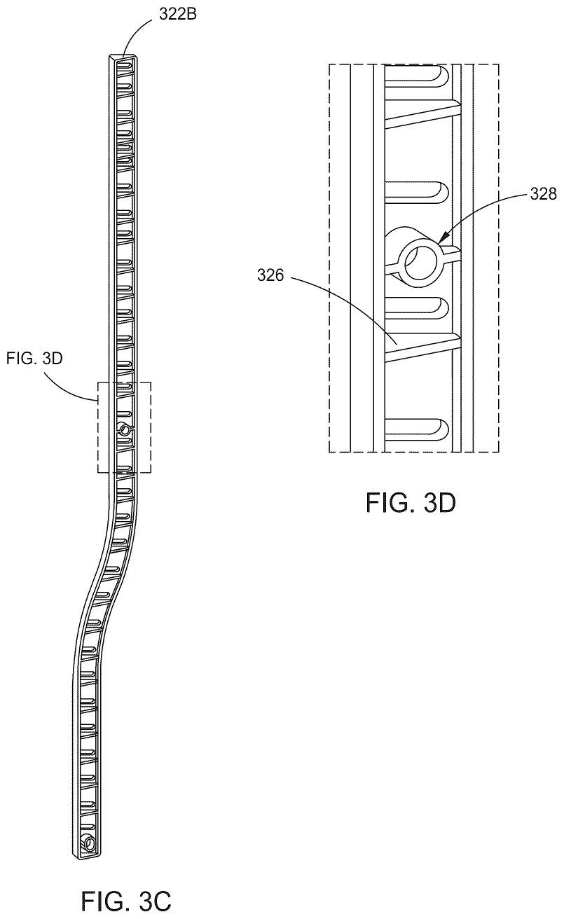

FIG. 3A illustrates an exploded perspective view of the fluidic module panel 114. The fluidic module panel includes a front panel 316 and a rear panel 318. FIG. 3B illustrates an exploded perspective of the rear panel 318. The rear panel 318 includes a first component 320, a second component 322, and a third component 324. The second component 322 includes a cover portion 322A and a rear portion 322B.

FIG. 3C illustrates the rear portion 322B of the second component 322. FIG. 3D illustrates a segment of the rear portion 322B. The rear portion 322B includes one or more light dividers 326 and one or more holes 328. The holes 328 are configured to receive a fastening component for assembling the fluidic module panel 114. The one or more light dividers 326 are configured to prevent light from one or more of the indicator lights 206 from interfering with another of the one or more indicator light 206.

The fluidic module panel 114 may include transparent plastic. The transparent plastic may be molded with in-mold decoration (IMD) film layer that includes ink layer to make portions the fluidic module panel 114 opaque, while other portions are translucent to allow for the one or more indicator lights 206 to be viewed.

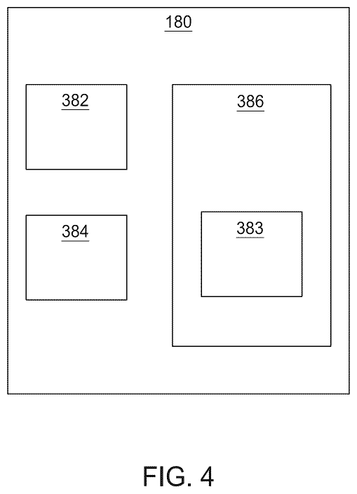

FIG. 4 illustrates a schematic of the controller 180. The controller 180 of the ophthalmic surgical system 100 includes a memory 386, a central processing unit (e.g., a processor 382), and support circuitry 384. The memory 386 includes executable instructions 383. The processor 382 is in data communication with the memory 386 and is configured to execute the instructions 383 in the memory 386. In some embodiments, the controller 180 may include a graphical user interface (GUI) to enable the user to manipulate the operations of the controller 180.

The controller 180 is configured to control the various components of the ophthalmic surgical system 100. The controller 180 can be one of any form of a general purpose computer processor that can be used in an industrial setting for controlling various chambers and sub-processors. The controller 180 can use any suitable memory 386, such as random access memory, read only memory, floppy disk drive, compact disc drive, hard disk, or any other form of digital storage, local or remote. Support circuitry 384 may be coupled to the controller 180 for supporting the ophthalmic surgical system 100. Bidirectional communications between the controller 180 and various other components of the ophthalmic surgical system 100 are handled through numerous signal cables collectively referred to as signal buses. In some embodiments, the controller 180 may control the brightness and operation of the task light 103A (e.g., each of the one or more lights of the task light 103A may be independently turned on, off, or have the brightness level adjusted by way of the GUI). The ability to adjust the brightness of the task light may enable the reduction of excess light in a dimly lit surgical environment.

The GUI may be operable to control the task light 103A. For example, a user may turn on the task light 103A with a quick tap on the task light icon on the GUI. A sustained press of the task light icon may bring up a sub-menu to operate the first light, the second light, or the third light independently. The GUI may include a graduated bar to control the brightness setting.

The controller 180 can be configured to cause the processor 382 to execute the instructions 383 of the memory 386. Executing the instructions 383 of the memory 386 may include causing the ophthalmic surgical system 100 to perform a method (e.g., method 500).

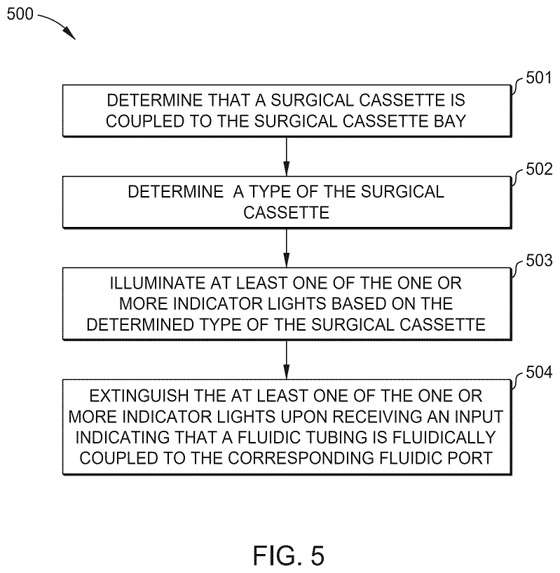

FIG. 5 illustrates a flow diagram of a method 500 of configuring a surgical console (e.g., an ophthalmic surgical system 100) for an ophthalmic surgical procedure. The ophthalmic surgical system 100 includes controller 180. The controller 180 includes a processor 382 and a memory 386, which may include instructions 383 for the method 500. The processor 382 is configured to execute the instructions 383 from the memory 386.

At operation 501, the processor 382 determines that a surgical cassette 210 is coupled to the surgical cassette port. At operation 502, the processor 382 determines a type of the surgical cassette 210. In some embodiments, the processor 382 is configured to scan the identifier of the surgical cassette 210 to determine the type. The identifier may be a bar code, a QR code, RFID, or other identifying feature of the surgical cassette 210.

At operation 503, the processor 382 illuminates at least one or more indicator lights 206 of the ophthalmic surgical system 100 based on the type of the surgical cassette 210. The one or more indicator lights 206 may include one or more LEDs. The indicator lights 206 may be configured to be different colors that correspond to different fluidic tubing 211.

At operation 504, the processor 382 extinguishes at least one or more of the indicator lights of the ophthalmic surgical system 100 upon receiving an input indicating that fluidic tubing is fluidically coupled with the corresponding fluidic port 208. For example, the indicator light 206 corresponding to a fluidic port 208 configured to receive an aspiration tube connector 212A may be extinguished upon receiving the aspiration tube connector 212A in the fluidic port 208.

In summation, the embodiments of the present disclosure provide an ophthalmic surgical system. A fluidics module of the ophthalmic surgical system has a surgical cassette bay configured to receive a surgical cassette. One or more indicator lights may correspond to one or more fluidic ports on the fluidics module. A memory of a controller of the ophthalmic surgical system includes executable instructions based on a surgical cassette received by the ophthalmic surgical system. A processor is configured to execute the instructions to cause illumination of least one of the one or more indicator lights based upon a determined type of surgical cassette. The indicator light corresponds to a fluidic port that is configured to receive a fluidics tube. When the fluidics port has received the fluidic tube, the indicator light is configured to extinguish. The one or more indicator lights make preparation of the ophthalmic surgical system in surgical environments more efficient and accurate, without introducing excess light into the surgical environment.

The above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. A system for ophthalmic surgical procedures, comprising:
a fluidics module, comprising:
a surgical cassette bay configured to receive a surgical cassette; and
one or more indicator lights corresponding to one or more fluidic ports on the fluidics module; and
a controller, the controller comprising:
a memory comprising executable instructions; and
a processor in data communication with the memory and configured to execute the instructions to cause the system to:
determine that a surgical cassette is coupled to the surgical cassette bay;
determine a type of the surgical cassette; and
illuminate at least one of the one or more indicator lights based on the determined type of the surgical cassette.

2. The system of claim 1, wherein the processor is further configured to execute instructions to cause the system to extinguish the at least one of the one or more indicator lights upon receiving an input indicating that a fluidic tubing is fluidically coupled with a corresponding fluidic port included in the one or more fluidic ports.

3. The system of claim 1, wherein the fluidic tubing includes at least one of an aspiration tubing or infusion tubing.

4. The system of claim 3, wherein a first indicator light of the one or more indicator lights corresponds to the aspiration tubing.

5. The system of claim 3, wherein a second indicator light of the one or more indicator lights corresponds to the aspiration tubing.

6. The system of claim 1, further comprising a task light housing and a task light.

7. The system of claim 6, wherein the task light includes a first light, a second light, and a third light.

8. The system of claim 6, wherein the processor is further configured to control an operation and a brightness of the task light.

9. The system of claim 1, wherein the processor is further configured to determine the type of the surgical cassette based on an identifier.

10. The system of claim 9, wherein the identifier includes a bar code, a quick-response (QR) code, or a radio frequency identification (RFID) tag.

11. A method of configuring a surgical console for an ophthalmic surgical procedure, comprising:
determining that a surgical cassette is coupled to a surgical cassette bay;
determining a type of the surgical cassette; and
illuminating at least one indicator light based on the determined type of the surgical cassette.

12. The method of claim 11, further comprising extinguishing the at least one indicator light upon receiving an input indicating that a fluidic tubing is fluidically coupled with a fluidic port corresponding to the at least one indicator light.

13. The method of claim 11, wherein the surgical cassette includes an identifier corresponding to the type of the surgical cassette.

14. The method of claim 13, wherein the identifier includes a bar code, a quick-response (QR) code, or a radio frequency identification (RFID) tag.

15. The method of claim 12, wherein the fluidic tubing includes at least one of an aspiration tubing or infusion tubing.
